(19) United States
(12) Patent Application Publication
(10) Pub. No.: US 2004/0050384 A1
(43) Pub. Date: Mar. 18, 2004

(54) Fire-resistant containers made using inorganic polymer material

(76) Inventors: Lawrence Stein, Los Angeles, CA (US); Marc R. Russak, Los Angeles, CA (US); Marc Hutson Montroy, Los Angeles, CA (US); Andrew Foden, Hamilton, NJ (US)

Correspondence Address: Dennis M. Flaherty, Esq., Ostrager Chong & Flaherty LLP, 30th Floor, 825 Third Avenue, New York, NY, 10022-7519, US

(21) Appl. No.: 10/245,558

(22) Filed: Sep. 17, 2002

(51) Int. Cl. A61M 11/00

(52) U.S. Cl. 128/200.23

(57) ABSTRACT

Fire-resistant containers made with inorganic polymer material. One type of container comprises a hollow body in the form of a matrix of inorganic polymer material. Other types of containers comprise a shell of non-inorganic polymer having interior or exterior surfaces treated with inorganic polymer material. Optionally, the hollow body further comprises filler material bound in the inorganic polymer material. For example, the filler material may comprise wound filaments. Some types of containers, e.g., munitions containers and firesafes, comprise a hollow body having an opening covered by a door, lid, or other closure device, the hollow body and closure device comprising inorganic polymer material.

### BACKGROUND OF THE INVENTION

This invention generally relates to fire-resistant containers and methods of improving the fire resistance of containers. In particular, this invention relates to a highly fire-resistant class of containers for the storage of any substance that is potentially dangerous when it comes in contact with heat or flames, or that is valuable and needs particular safeguarding against heat and flame.

The categories of containers that may advantageously be made from fire-resistant materials include, but are not limited to, the following: containers for compressed substances, containers for storing explosive or hazardous substances, munitions containers, and safes for valuables.

For example, cylinders manufactured to contain compressed gas or compressed liquid have been in existence for over 200 years. Originally, compressed gas cylinders were introduced in order to contain and transport gasses. A brief survey of the history of the self-contained breathing apparatus (SCBA) will serve to illustrate the evolution of containers manufactured for storing and transporting of compressed materials.

Early firefighters had to face not only fire and the effects of heat with little or no water, but also the debilitating effects of smoke with nothing at all to protect them. Firemen could not effectively operate under the heavy smoke conditions encountered during structure fires. The breathing difficulties encountered by firemen were so pronounced that they often grew long beards, wet them and clinched the hairs between their teeth in an effort to create a filter to ease breathing.

The first successful American self-contained breathing apparatus (SCBA) was known as the Gibbs. Experiments with this unit began in 1915 and by 1918 they were being manufactured by Edison Laboratories in Orange, New Jersey. Toward the end of World War II, Scott Aviation was manufacturing breathing equipment that allowed aircrews to operate at extreme altitudes. The products were then adapted and used by firefighters. Protective breathing apparatuses have remained the most important piece of equipment used by every firefighter, and they are also crucial for other fields, such as aviation. Failure by firefighters to use this equipment has resulted in failed rescue attempts, firefighter injuries and fatalities. Non-use of this equipment by aviators could yield similar results.

Because the cylinder must be strong enough to withstand the high pressure of compressed air, the cylinder itself constitutes the main weight of the breathing apparatus. The weight of the air cylinders varies with each manufacturer, and depends on the material used to fabricate the cylinder. Manufacturers offer cylinders for various uses and therefore in various sizes and materials.

For many years, most SCBAs were made of steel. Made of relatively thin rolled stock and welded at the seams, most steel cylinders are heavy to lift and use, costly to transport, and prone to rust and corrosion. Eventually steel SCBAs were replaced with a better alternative. Cold indirect extrusion techniques led to the development of a process for mass producing seamless aluminum cylinders, which are up to 30 percent lighter than their steel counterparts.

The lighter-weight aluminum cylinders were slowly phased out with the advent of carbon fiber composite cylinders. These composite cylinders, which utilize thin aluminum liners, can accept higher charging pressure and thus enable end users to carry more gas in a given volume and work more safely while carrying high-pressure gases in extreme environments. Today, the majority of SCBAs used by firefighters are made from of carbon fiber composite material that surrounds a relatively thin liner, generally made of aluminum. This liner is responsible for maintaining the shape of the cylinder and provides about 10% of the strength needed to withstand the high pressure inside the cylinder. The carbon filament provides the remaining 90% of the cylinder strength.

This known SCBA is manufactured as follows. First, the carbon filament is dipped in an organic resin, and then the impregnated filaments are wound around the liner in a scientifically calculated pattern. The liner is made of aluminum. The organic resin holds the carbon filaments, permanently bonding the filaments to the cylinder. In essence, the resin permanently glues the carbon filaments to the cylinder liner, and has the added benefit of adding strength to the structure.

The wrapping of the resin-impregnated filament around the cylinder occurs on a filament-winding machine. This machine resembles a lathe, with one or more arms around the outside that distribute the filament/resin over the slowly rotating cylinder. These machines are specifically calibrated to apply even layers of filament to ensure that the pressure inside the cylinder is evenly distributed, thus allowing for optimal containment and reducing the risks of rupture and explosion. To further reduce this risk, compressed substance cylinders typically have some sort of safety mechanism, such as a rupture disk, that bleeds off the contents slowly when the internal pressure exceeds a predetermined threshold, thereby reducing the likelihood of an explosion.

In the case of SCBAs intended for use by firefighters, the proximity to heat and flame makes it highly desirable that SCBAs be made of fire-resistant materials. Surprisingly, the epoxy resin currently used in the manufacture of carbon fiber SCBA cylinders is not fire resistant. Inadequate fire resistance afflicts the vast majority of compressed substance containers in the marketplace. Cylinders made of composite material are actually flammable, while cylinders made of aluminum or steel, although they are not flammable, have efficient heat transfer properties that facilitate rapid increase in the pressure inside the container, be it an SCBA or other type of container.

There is a need to improve the fire- and heat-resistant qualities of containers designed to store and transport compressed liquids or gases. There is also a need to improve the fire- and heat-resistant qualities of containers designed to store and transport non-compressed materials, such as hazardous chemicals, explosive materials and munitions. There is a further need to improve the fire and heat resistance of safes and other containers for holding currency, securities, artwork, jewelry or other valuables.

### BRIEF DESCRIPTION OF THE INVENTION

The invention is directed to fire-resistant containers made with inorganic polymer material. One type of container comprises inorganic polymer material in the form of a hollow body. Other types of containers comprise a shell of non-inorganic polymer having interior or exterior surfaces treated with inorganic polymer material. For example, an SCBA can be constructed as a liner with an outer coating of inorganic polymer material. Optionally, the hollow body further comprises filler material bound in the inorganic polymer binder material. For example, the filler material may comprise wound filaments. Some types of containers, e.g., munitions containers and firesafes, comprise a hollow body having an opening covered by a door, lid, or other closure device, the hollow body and closure device comprising inorganic polymer material.

One aspect of the invention is a hollow body comprising an inorganic polymer material. In some embodiments, the hollow body further comprises a lining material substantially enclosed by an inorganic polymer material or coated with inorganic polymer material. Optionally, the hollow body further comprises filler material bound in the inorganic polymer binder material. For example, the filler material may comprise wound reinforcing fibers or other additives to permit processing and enhance performance in service.

Another aspect of the invention is a self-contained breathing apparatus comprising an air tank for storing and delivering air under pressure, a face mask to be received over a user's face, a hose connecting the air tank to the face mask, and a regulator for reducing the pressure of air delivered from the air tank to a breathable pressure at the face mask, wherein the air tank comprises a hollow body wrapped by filaments bound in an inorganic polymer binder material.

A further aspect of the invention is a method of improving the fire resistance of a container, comprising the step of applying inorganic polymer material to a surface of the container.

Another aspect of the invention is a fire-resistant container comprising a hollow body having an interior surface and an exterior surface, wherein at least one of the interior and exterior surfaces is substantially covered with inorganic polymer material.

An additional benefit of using inorganic resin as opposed to organic resin in a manufacturing process is that the costs and hazards associated with the cleanup phase of the manufacturing process are reduced. In particular, inorganic polymeric binder can be cleaned up using water instead of the more costly and potentially harmful solvents typically used to dissolve organic resins.

Other aspects of the invention are disclosed and claimed below.

### BRIEF DESCRIPTION OF THE DRAWINGS

FIG. 1 is a drawing showing a side elevational view showing a typical self-contained breathing apparatus, with a person's head being shown in phantom.

FIG. 2 is a drawing showing a partially sectioned view of an air tank with ancillary apparatus for coupling a hose to the air tank.

FIG. 3 is a drawing showing a sectional view of a container wall comprising a liner material and an inorganic polymer binder material with embedded wound filaments in accordance with one embodiment of the present invention.

### DETAILED DESCRIPTION OF THE INVENTION

In the embodiments of the invention, the fire resistance of containers is enhanced by incorporating an inorganic polymer material. Existing containers that are not fire-resistant can be treated with inorganic polymer material. New fire-resistant containers can be manufactured by casting inorganic polymer material to form a hollow body. Alternatively, new fire-resistant containers can be manufactured by making a liner and then applying inorganic polymer material to the exterior of the liner. The inorganic polymer material may have filler material bound therein, e.g., wrapped filaments for increasing the strength of the container.

In one embodiment not shown in the drawings, a container may be constructed from inorganic polymer material only. If such a container has a door, lid or other closure device, that closure device is also made of inorganic polymer material. More specifically, alkali-activated silico-aluminate inorganic polymeric binder can be used. In this specification, the term “alkali-activated silico-aluminate polymeric binder” (or simply “inorganic polymer”) refers to an inorganic binding material comprising alumino-silicate oxide, or other similar materials known to persons skilled in the art. Inorganic polymeric binders were developed for use in preparing high-strength masonry products, such as tiles.

An inorganic polymer is a reactive aluminosilicate binder obtained by mixing two or more components that remain inert when used separately. Typically, one component is a liquid and the other component is a solid. When mixed together, the components react to form a polymeric network. Heat and/or pressure may be required to propagate the polymeric reaction. For example, GEOPOLYMITE 50™ is a commercially available inorganic polymer manufactured by Geopolymere S.A.R.L., a French company. GEOPOLYMITE 50™ consists of two parts, A and B, which are combined in equal proportions just prior to use. Part A is liquid and part B is in powder form. The chemical analyses of the two parts is shown in Table 1 (taken from U.S. Pat. No. 4,859,367):

CHEMICAL ANALYSIS OF GEOPOLYMITE 50

| | Part A | Part B |
|---|---|---|
| SiO₂ | 20.95 | 30.22 |
| Al₂O₃ | — | 25.30 |
| Fe₂O₃/TiO₂ | — | 1.10 |
| K₂O | 25.98 | 0.63 |
| CaO | — | 29.00 |
| MgO | — | 2.76 |
| F | — | 10.94 |
| H₂O | 53.03 | — |
| Total | 99.96 | 99.95 |

The foregoing chemical analysis of a known inorganic polymer is presented for illustrative purposes only and is not meant to suggest that other inorganic polymer materials cannot not be used to practice the present invention.

Another example of a category of inorganic polymer having application in the present invention are the modified alkali silicate compositions disclosed in International Publ. No. WO 02/24597 A2, entitled “Inorganic Matrix Compositions, Composites and Process of Making the Same.”

One example of a container that may be constructed from inorganic polymer material is a munitions container, e.g., a container for a grenade, rocket or other explosive projectile. A known type of munitions container comprises a circular cylindrical tube for a single or several munitions units. The chamber of the container may have inner support elements, such as foamed plastic material, cardboard or rubber, for cushioning the munitions during transport. The tube is closed at the bottom and has a screw-off top or hinged lid.

In accordance with one embodiment of the present invention, a munitions container of the foregoing type can be made of inorganic polymer material with or without filler material. For example, a circular cylindrical tube with a closed bottom can be formed by casting the inorganic polymer material in a suitable mold. The mold may be of the type that is disassembled to remove the finished product. Likewise a screw-off top can be cast in a suitable mold. The tube is designed to receive the munitions along with suitable means for supporting the munitions in a central position out of contact with the inorganic polymer tube. When the top of the container is screwed on, the munitions are safely enclosed in a fire-resistant container.

In accordance with an alternative embodiment, a circular cylindrical tube can be formed by wrapping inorganic polymer resin-wetted filaments around a circular cylindrical mold using a filament winding machine. Exemplary filaments that can be used in this process include, but are not limited to, filament made of carbon, fiberglass, silicon carbide, aramid (e.g., Kevlar), or other fibers known to persons skilled in the art. A method and an apparatus for making fiber-reinforced piping are disclosed in U.S. Pat. No. 5,031,846, the disclosure and drawings of which are fully incorporated herein by reference. One or more layers of resin-wetted filament can be wrapped around the circular cylindrical mold to form a tube that is open at both ends. The closures for the ends are separately molded, with fiber reinforcement if necessary, and attached to the tube. When more than one layer of filament is wound, the filaments of adjacent layers are preferably laid in different directions or orientations. The inorganic polymer resin-impregnated layers of filament are then cured and hardened to form a composite tube. The finished composite is then separated from the mold.

In accordance with a further embodiment of the invention, the filament winding process is repeated, except that instead of a circular cylindrical mold, the filament is wrapped around a circular cylindrical liner that will not be removed and will form part of the final munitions container. Such a liner may be formed from metal, ceramic or plastic. The bottom of the liner may be closed by a hemisphere of the same material. The top may be closed by a hinged lid, a screw-off cover, or any other suitable closure.

As can be appreciated, the inorganic polymer in the present invention can be formed by any fabrication method capable of applying the necessary temperature and/or pressure to form the inorganic polymer into the desired form. Typical processes include compression molding, pultrusion, wet lay-up, autoclave vacuum bag processing, non-autoclave vacuum bagging, vacuum infusion or powder infusion, resin transfer molding, extrusion, injection molding, casting, spin casting, trapped elastomer molding, and like processes known to persons skilled in the art.

In accordance with another embodiment of the invention, a cylinder containing gas or liquid may be made of inorganic polymer material with or without filler material and with or without liner material. Some applications for fire-resistant cylinders include, but are not limited to, a compressed air cylinder for a self-contained breathing apparatus, a cylinder for containing compressed liquid natural gas, a compressed air cylinder for a paintball gun; and cylinders for home-oxygen therapy.

A primary weakness of many known containers that store compressed material is susceptibility to heat and flame. In particular, cylinders made using organic resins have increased fire and smoke hazards due to the combustibility of the organic resin. By replacing the typical epoxy resins found in compressed gas cylinders and other similar apparatuses with an inorganic resin, the container will be able to withstand greater exposure to direct flame and high temperatures. By way of example, in laboratory tests a 1-cm-thick slab of carbon fiber impregnated with inorganic polymer resin was able to withstand direct flame from a torch at 1,832° F. for 30 minutes. After a full half hour of exposure to this flame, not only did the slab fail to ignite, but the side of the slab opposite the flame registered a temperature of only 356° F.

Reference will now be made to the drawings, in which similar elements in different drawings bear the same reference numerals. For the purpose of illustration, a self-contained breathing apparatus in accordance with one embodiment of the invention will be described.

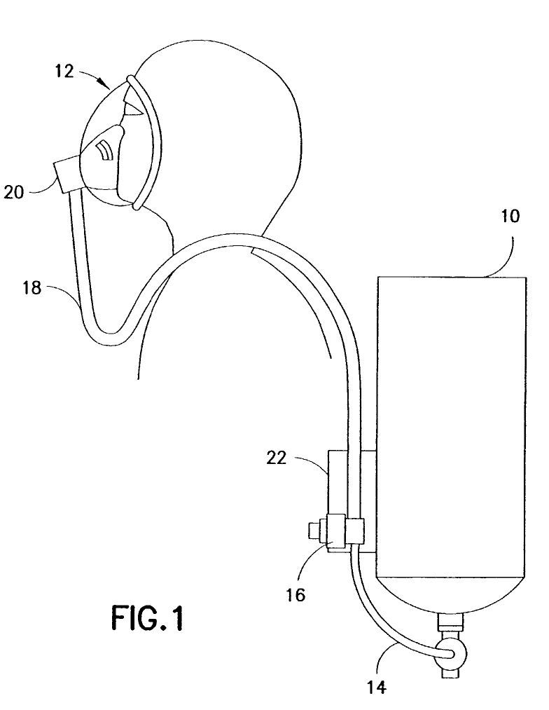

A typical SCBA is depicted in FIG. 1. This apparatus comprises a face mask 12 having a pressure regulator 20 thereon, and a hose 18 connecting the regulator 20 to a pressure reducer 16. The pressure reducer 16 is connected to the tank of air 10 by a high-pressure hose 14. A battery-powered control box 22 is connected to the pressure reducer 16 by a tube (not visible in FIG. 1) that carries air at tank pressure. A transducer within the control box 22 receives the full pressure of the tank and converts the pressure into an electrical output to a display device not shown.

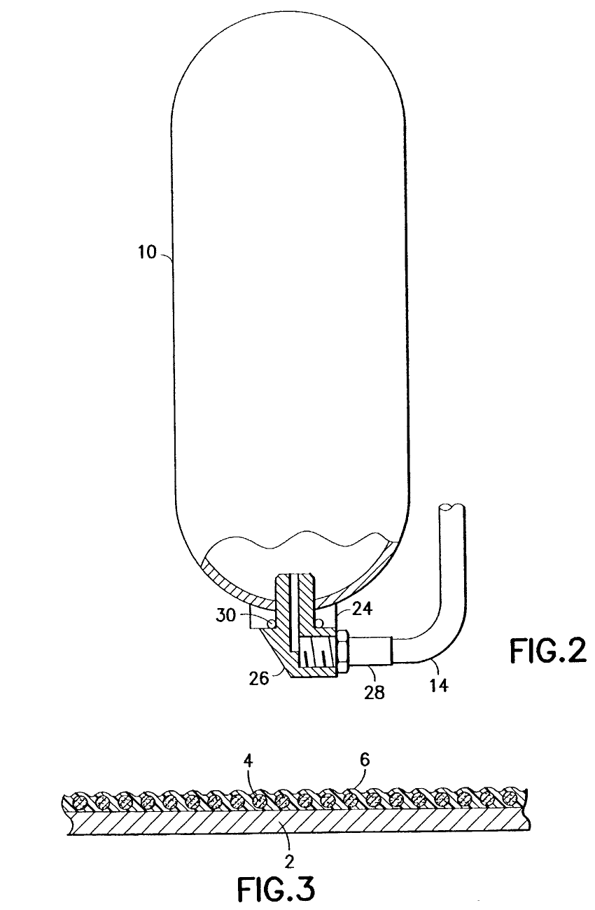

FIG. 2 shows a conventional means for coupling an air cylinder or tank 10 to a high-pressure hose 14. The hose 14 is connected to the cylinder 10 at a cylinder inlet 26 by means of the coupling 28, which has a threaded end that screws into a threaded bore of the cylinder inlet. The cylinder inlet has a passageway that connects the interior of the tank 10 to the open threaded end of the coupling 28. An O-ring 30 is placed between the cylinder inlet 26 and a neck 24 of the cylinder to prevent leakage of air therefrom.

FIG. 2 depicts an air cylinder made of metal. As previously discussed, it is known to manufacture air cylinders for SCBAs comprising a metal (e.g., aluminum) liner with a carbon fiber-reinforced layer of organic resin. The carbon fibers or filaments are wetted with the organic resin and then wrapped around the metal liner using conventional filament winding technology.

In accordance with an embodiment of the present invention, inorganic polymer resin is substituted for the organic resin in the manufacturing process. The basic structure of this composite cylinder is shown in FIG. 3. A metal liner 2 in the shape of a circular cylinder closed at both ends by hemispheres is wrapped with carbon filaments 4 wetted with inorganic polymer binder material. When the inorganic polymer binder material is cured and hardened, an inorganic polymeric matrix 6 is formed that binds the carbon filaments 4 in place. Although FIG. 3 shows only one layer of filaments wrapped around the liner, more than one layer of filament can be wrapped around the liner, as previously described.

It should also be appreciated that the inorganic polymer binder material, in addition to filler material, may also have other ingredients, such as: a surfactant to facilitate wetting of filler material; a thickening agent that provides nucleation sites for silicate growth.

The concept of the invention, as embodied above, may be expanded to encompass other types of containers comprising inorganic polymer material for improving fire resistance. For example, the invention has application in a fire-resistant safe comprising a door with a combination lock. The door and the walls of the safe can be made of inorganic polymer material. Alternatively, the interior or exterior surface of a safe can be coated with inorganic polymer material to render the safe fire-resistant. Since safes come in all sizes, the same principle applies to safes as large as bank vaults and safes as small as lock boxes.

In addition, the invention may be embodied as a storage tank for any substance that is explosive, flammable, toxic, or otherwise hazardous. Such a storage tank may have walls made of inorganic polymer material or walls treated with inorganic polymer material. The tank may be penetrated by ancillary components for filling and/or emptying the tank, or a release valve or rupture disk or diaphragm for relieving any undesirable buildup of pressure within the tank. In particular, the tank may have a diaphragm designed to burst at a predetermined pressure differential between the pressure inside the tank and the pressure outside the tank.

While the invention has been described with reference to various embodiments, it will be understood by those skilled in the art that various changes may be made and equivalents may be substituted for elements thereof without departing from the scope of the invention. In addition, many modifications may be made to adapt a particular situation to the teachings of the invention without departing from the essential scope thereof. Therefore it is intended that the invention not be limited to the particular embodiment disclosed as the best mode contemplated for carrying out this invention, but that the invention will include all embodiments falling within the scope of the appended claims.

1. A hollow body comprising an inorganic polymer material.

2. The hollow body as recited in claim 1, further comprising a lining material enclosed by said inorganic polymer material.

3. The hollow body as recited in claim 2, further comprising filler material bound in said inorganic polymer material.

4. The hollow body as recited in claim 3, wherein said filler material comprises reinforcing filaments.

5. The hollow body as recited in claim 2, wherein said lining material is selected from the group consisting of metals, ceramics and plastics.

6. The hollow body as recited in claim 4, wherein said filaments are made of carbon.

7. The hollow body as recited in claim 4, wherein said filaments are made of fiberglass.

8. The hollow body as recited in claim 4, wherein said filaments are made of aramid.

9. The hollow body as recited in claim 4, wherein said filaments are made of silicon carbide.

10. The hollow body as recited in claim 1, wherein said hollow body has an opening, further comprising a cover that closes said opening, said cover comprising an inorganic polymer material.

11. The hollow body as recited in claim 1, wherein said hollow body has an opening, further comprising an ancillary device inserted in said opening.

12. A self-contained breathing apparatus comprising an air tank for storing and delivering air under pressure, a face mask to be received over a user's face, a hose connecting said air tank to said face mask, and a regulator for reducing the pressure of air delivered from said air tank to a breathable pressure at said face mask, wherein said air tank comprises a hollow body reinforced by filaments bound in an inorganic polymer material.

13. A method of improving the fire resistance of a container, comprising the step of applying inorganic polymer material to a surface of said container.

14. The method as recited in claim 13, wherein said inorganic polymer material is applied on the exterior of said container, further comprising the step of wrapping the exterior of said container with a filament.

15. The method as recited in claim 14, further comprising the step of dipping said filament in inorganic polymer material before wrapping, wherein inorganic polymer material is carried by said filament during wrapping.

16. The method as recited in claim 13, wherein said inorganic polymer material is applied on interior surfaces of said container.

17. A fire-resistant container comprising a hollow body having an interior surface and an exterior surface, wherein at least one of said interior and exterior surfaces is substantially covered with inorganic polymer material.

18. The fire-resistant container as recited in claim 17, wherein said hollow body comprises a cylinder having its exterior surface substantially covered with inorganic polymer material, further comprising filaments wrapped around said cylinder and bound by the inorganic polymer material.

19. The fire-resistant container as recited in claim 18, wherein said cylinder comprises a neck penetrated by a bore, further comprising a cylinder inlet installed in said bore.

20. The fire-resistant container as recited in claim 17, wherein said hollow body comprises a lid that can open and close, further comprising munitions stored in said hollow body.

21. The fire-resistant container as recited in claim 17, wherein said hollow body comprises a door that can open and close, further comprising a lock having locked and unlocked states, said door being openable only when said lock is in said unlocked state.

22. The fire-resistant container as recited in claim 17, wherein said hollow body comprises a screw top that can open and close.

23. The fire-resistant container as recited in claim 17, wherein said hollow body comprises a tank and a diaphragm designed to burst at a predetermined pressure differential between the pressure inside said tank and the pressure outside said tank.
